(19) United States
(12) Patent Application Publication
(10) Pub. No.: US 2006/0105000 A1
(43) Pub. Date: May 18, 2006

(54) Compositions for treating infected skin and mucous membrane comprising an anti-microbial agent and an essential oil

(75) Inventor: Doron I Friedman, Karme Yosef (IL)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: J.P.M.E.D. LTD, KARME YOSEF (IL)

(21) Appl. No.: 10/535,961

(22) PCT Filed: Nov. 19, 2003
(86) PCT No.: PCT/IL03/00980

§ 371 Date: May 20, 2005

(30) Foreign Application Priority Data
Nov. 21, 2002 (IL) 152993
Nov. 17, 2003 (IL) 158901

(51) Int. Cl. A61K 36/18; A61K 9/00

(52) U.S. Cl. 424/400; 424/725

(57) ABSTRACT

The invention provides a composition of matter for treating infected skin and mucousal membranes, said composition comprising at least one anti-microbial drug; and at least one essential oil, in combination with a substantially, alcohol-free carrier system, said carrier being selected from a liquid carrier or a semi-solid carrier, said carrier system being selected from isotonic system and a moderately hypertonic system.

The present invention relates to a composition for treating infected skin and mucousal membranes. More particularly, the present invention relates to a composition for treating microbially infected skin and mucousal membranes including the treatment of wounds and skin ulcers, comprising a mixture of at least one anti-microbial drug and at least one essential oil, in a liquid or semi-solid carrier delivery system. Preferably said compositions are stabilized with at least one inactive ingredient or excipient which is non-cytotoxic at the concentration used and which does not inhibit wound-healing, and which is isotonic or moderately hypertonic.

### BACKGROUND OF THE INVENTION

Multi antibiotic resistance of pathogenic bacteria is becoming an obstacle for effective infection treatment. Mucous and skin infections are in many cases hard to treat since they often involve multi bacterial, yeast and fungal infections. Mucous and skin infections are also commonly associated with inflammation, ulceration and bleeding. There is a need for a more complex product to provide a solution to the many factors encompassing the infected mucous or skin status and clinical manifestations.

Many of current pharmaceutical and cosmetic inactive ingredients or excipients are harsh chemicals which are irritating to already affected tissue and inhibit wound healing and new tissue generation by denaturing growth factors involved in tissue healing or are cytotoxic to the fibroblasts or keratinocytes.

Mucositis, Vaginitis, Anal fissure, Pressure sores, Dermatitis, Otitis, Gingivitis and Periodontitis and skin ulcers, are all prone to multi-microbial infection and inflammation, and involve difficult to cure conditions, because of the enormous number of germs in the affected area.

There is a need for products that will have good anti-microbial activity, that will simultaneously affect bacteria, yeast, fungi and viruses, that will not be susceptible to bacterial resistance and will be innocuous to affected tissue, and which will not inhibit the natural wound healing process.

Mucositis

Mucositis is an inflammation and ulceration of the lining of the mouth, throat or gastrointestinal tract most commonly associated with chemotherapy or radiotherapy for cancer.

Common manifestations of mucositis include ulcerations, redness, and swelling in the mouth as well as cramping, diarrhea and bleeding. In more severe cases, mucositis can be extremely painful, preventing the patient from eating and necessitating hospitalization for hydration, narcotic pain medication, and/or total parenteral nutrition. The destruction of the protective mucous membrane can also place the patient at a serious risk of infection.

The consequences of mucositis can be significant. Mucositis is often a dose-limiting toxicity of chemotherapy and radiation therapy, leading to reductions or delays in chemotherapy or irradiation doses. Dose-limiting toxicities such as mucositis are a major concern for oncologists because they adversely impact the curative potential of the patient's primary therapy. In addition, mucositis may lead to dehydration, malnutrition, or infection, all of which compromise the desired treatment plan.

There are presently no pharmaceutical agents available on the market to prevent or treat mucositis. Severe mucositis necessitates a delay in the chemotherapy schedule or reduction of the dose as well as treatment of complications such as pain, dehydration, malnutrition, and infection.

Unmet needs in the area of mucositis include therapies that prevent or reduce the severity, duration, and symptoms of mucositis so the patient's chemotherapy or radiation regimen can be maintained or intensified. Clinicians also desire a therapy that reduces hospitalization, narcotic use, or the need for nutritional support.

Vaginitis

The three most common vaginal infections are: Bacterial vaginosis, Candidiasis, or “yeast infection” and Trichomoniasis. It is common that more than one infection is present.

Anal Fissure

Anal fissure tends to become ulcerated or infected because of the enormous number of germs in this area; an anal fissure will always get infected, so there is local inflammation.

An anal fissure is a superficial linear tear in the anoderm most commonly caused by passage of a large, hard stool. This tear is distal to the dentate line. Anal fissures are among the most common anorectal disorders in the pediatric population; however, adults also are affected.

The majority of anal fistulas originate in anal crypts, which become infected with abscess formation. When the abscess is opened or ruptures, a fistula is formed.

Skin Ulcers

A contaminated wound will heal, an infected wound will not. Stage 2, 3 and 4 pressure ulcers should all be considered as colonized with bacteria. Infected skin ulcer may result from pressure, arterial insufficiency, venous stasis, diabetic, traumatic or burns.

Pressure Sores

Pressure sores that become infected heal more slowly and can spread a dangerous infection to the rest of the body.

The antibiotic for pressure sores should be effective against gram-negative, gram-positive, and anaerobic organisms (e.g., silver sulfadiazine, triple antibiotic). Monitoring allergic sensitization and other adverse reactions is necessary due to many non-curing situations.

Oral Ulcers

There is essentially no known cause or cure of intra-oral ulcers. These ulcers can be extremely painful to patients, and generally persist for seven to ten days. While the etiologies of oral aphthae, or canker sores, are quite varied, the central concern is the severe pain they cause. This pain affects the quality of life for millions of individuals. It, is believed that the pain related to the oral ulcerative lesions is made more severe by the secondary infections caused by the prevailing oral bacteria.

One currently used common treatment for intra-oral ulcers is the use of acyclovir, an antiviral agent effective in treatment of certain forms of herpes. Acyclovir is available from Glaxo Wellcome under the tradename Zovirax. Zovirax consists essentially of acyclovir in a polyethylene glycol base and is available as an ointment or rinse. This product approaches the problem of ulcers based on the hypothesis that such ulcers or lesions are viral in nature. Another recent treatment is use of a product called Aphthasol, available from the Block Drug Company. Aphtasol consists of amlexanox, an antihistamine, in an adhesive paste. This treatment is based on the hypothesis that oral ulcers and lesions are caused by an autoimmune or allergic response of the body. However, none of the above products has proven to be effective in reliably reducing the pain associated with the ulcer while simultaneously speeding the healing process and preventing secondary infections.

Gingivitis

Gingivitis is a disorder involving inflammation of the gums. Gingivitis is caused by the long-term effects of plaque deposits. Plaque is the sticky material that develops on the exposed portions of the teeth, consisting of material such as bacteria, mucus, and food debris. It is a major cause of dental caries. Un-removed plaque mineralizes into a hard deposit called calculus (tartar) that becomes trapped at the base of the tooth. Plaque and calculus cause mechanical irritation and inflammation of the gingiva. Bacteria, and the toxins produced by the bacteria, cause the gums to become infected, swollen, and tender.

Severe gingivitis conditions end up in finally acute necrotizing ulcerative gingivitis, Which can be life threatening.

The goal of treatment is reduction of gingival inflammation. Daily oral hygiene may include tooth brushing and oral rinse. Common toothpastes and mouth rinses comprise antiseptic agents such as Chlohexidine, Cetyl pyridinium chloride, Essential oils such as Menthol, Thymol, Methylsalycilate and Eucaliptol, usually in a hydro-alcoholic solvent carrier. Commercial mouth rinses are hypertonic or contain significant concentration of gingivitis inhibiting agents or compositions.

It is an object of the present invention to provide effective anti-septic and anti-bacterial composition that does not comprises ingredients that inhibit gingival healing process and do not inhibit inflammation recovery.

Periodontitis

Periodontitis is a dental disorder that results from progression of gingivitis, involving inflammation and infection of the ligaments and bones that support the teeth. Besides dentist intervention, periodontitis is treated by application of anti-bacterial agents such as Chlohexidine and Metronidazole, directly into periodontal pockets.

Otitis

Acute otitis media (OM) is a middle ear infection that may cause a change in the normal eardrum, which is located at the inner end of the ear canal. Otitis externa (OE) is an infection of the outside ear canal and/or opening to the ear and is commonly called “swimmer's ear.”

In OM, the infection can be caused by a virus or by bacteria. It may also be accompanied by allergies, enlarged adenoids, or a cold that causes blockage of the eustachian tube (the connection between the throat and middle ear that equalizes pressure).

In OE, the infection is often caused by scratching, inserting objects into the ear canal, or moisture, such as pool water. OE may also be caused by bacteria or fungi.

Dermatitis

Dermatitis is an inflammation of the dermis and epidermis (the skin). Symptoms include a rash, blisters, sores, lesions, itching or cracked skin. The term eczema is also used for this condition.

U.S. Pat. No. 5,213,615 discloses a dental material for the control of caries and paradentitis, which contains an active agent combination of thymol and/or carvacrol and chlorhexidine and/or the physiologically compatible salts thereof. The dental material can be a dental varnish or a material such as a dental cement and the like, which remains in the oral cavity for a long period and from which the active combination can diffuse out.

U.S. Pat. No. 4,693,888 discloses “a caries-preventive composition comprises an antibody obtained by immunizing a mammal with at least one antigen selected from the group consisting of Streptococcus mutans, its cell-wall fraction, fibrous substance fraction, glucosyltransferase fraction and protein antigen fraction, and a synergist selected from the group consisting of fluorine compounds, chlorhexidine and its salts, lytic enzymes, bacteriocins, glucosyltransferase inhibitors, proteases and dextranases”.

U.S. Pat. No. 6,352,711 discloses pharmaceutical compositions which comprise of an effective amounts of antimicrobials, anti-inflammatories, and antihistamines, to provide an ulcer medication which prevents secondary infections and promotes healing while providing immediate relief from pain. The composition may be used to treat a variety of ulcers including but not limited to intraoral aphthous ulcers and non-oral lesions.

U.S. Pat. No. 6,387,352 states that “Although chlorhexidine has been shown to be useful in the prevention of bacterial and fungal infection, there are no consistent findings in the value of chlorhexidine in reducing mucositis in cancer patients. It probably works on the secondary microbial initiation of already-affected tissue. The problem with its use is that, once mucositis starts, the alcohol content of chlorhexidine preparations makes it difficult for the patient to use even at one-half strength. It is difficult to force the patients who are experiencing severe pain and who are already on morphine to use something that increases their pain. Chlorhexidine was used as long as the patient could tolerate it—usually until the onset of mucositis.”

U.S. Pat. No. 6,458,777 discloses administration of anti-microbial agents in combination with “inflammatory cytokine inhibitor” which “result in an even more effective method for treating and preventing mucositis”.

None of the above-mentioned patents teaches the composition of antibiotic and essential oils for preventing or treating mucous or wound infections.

Recent scientific data suggests that alcohol may play a role in toxic and genotoxic biological effects. Consumers are therefore refraining from using products containing alcohol, especially for sensitive body organs such as for the oral cavity and babies' skin. Alcohol is an irritant to the skin, scalp, mucous membrane and gastrointestine. In the oral cavity alcohol has a foul taste, which is especially unpleasant for young and elderly people. Alcohol burns tissues in a way that delays tissue healing after skin traumas. Alcohol dehydrates the skin, mucous membrane and tissues, which in turn causes discomfort and pain. Therefore medical research is investing in finding alcohol free medicaments.

Chlorhexidine, an antimicrobial mouth rinse, has also been used extensively in the treatment and prevention of oral mucositis (Ferretti et al., 1990, Bone Marrow Transplan. 3:483-493; Weisdorf et al., 1989, Bone Marrow Transplan. 4:89-95). It has been noted however that the efficacy of chlorhexidine is significantly decreased in saliva, and that this compound is relatively ineffective against the Gram negative bacteria that tend to colonize the oral cavity in patients undergoing radiation therapy (Spijkervet et al., 1990, Oral Surg. Oral Med. Oral Pathol. 69:444-449). In addition, at least one study has shown that the use of chlorhexidine may be detrimental and result in a higher incidence of mucositis (Foote et al., 1994, J. Clin Oncol. 12:2630-2633).

Several studies have shown that the use of a vancomycin paste and antibiotic lozenges containing polymixin B, tobramycin and amphotericin B in patients undergoing myelo-suppressive chemotherapy or radiation therapy can result in a decrease in oral mucositis and in the incidence of sepsis due to alpha hemolytic streptococci (Barker et al., 1995, J. Ped. Hem. Oncol. 17:151-155; Spijkervet et al., 1991, In: Irradiation Mucositis, Munksgaard Press, pp. 43-50). Despite the clear need for therapeutic agents to treat oral mucositis, no drugs are currently approved for this indication. As a result, there is no standard treatment for this disorder.

The present invention is based on the discovery that combinations of at least one anti-microbial agent and at least one essential oil, formulated in a liquid or semi-solid delivery system that comprises only such ingredients that are non-toxic and are not wound healing inhibitors, at the concentration used, provide unexpected and highly effective Mucositis and Ulcers medications.

The present invention provides a medication, which prevents and treats infection, inflammation, and bleeding and promotes healing while simultaneously providing relief from pain for infected skin and mucousal membranes.

Moreover, in contrast to above-mentioned discussion of the harmful effect of chlorhexidine and its inappropriate use in mucositis, it has now been discovered that a mixture of chlorhexidine with essential oils in an alcohol-free formulation is beneficial to patients suffering from severe mucositis; is effective for curing the infection and inflammation and for reducing pain and improving swallowing difficulties. The alcohol-free chlorhexidine formula enables repeated usage, several times a day, since the formula is well accepted. It has now been further discovered according to the present invention that it is possible to prepare a mouthwash formula of bitter drugs such as chlorhexidine having improved taste, thereby improving patient's compliance. It has also been discovered that a medicated mouthwash composition of matter according to the present invention is effective in preventing and treating mucositis.

It has also been found that combining of at least one anti-bacterial or anti-septic drug with an essential oil or with a mixture of essential oils, when formulated in a system selected from an isotonic system and a moderately hypertonic system which system is substantially free of wound healing inhibitors and substantially free of cytotoxic agents, is unexpectedly more effective in treating external infectious conditions of skin and mucous, such as: Otitis, Anal fissure, Dermatitis, Gingivitis, Periodontitis and Mucositis, as mentioned above.

### DETAILED DESCRIPTION OF THE INVENTION

Thus, according to the present invention, there is now provided a composition of matter for treating infected skin and mucousal membranes, said composition comprising at least one anti-microbial drug; and at least one essential oil, in combination with a substantially, alcohol-free carrier system, said carrier being selected from a liquid carrier or a semi-solid carrier, said carrier system being selected from isotonic system and a moderately hypertonic system.

In preferred embodiments of the present invention, said carrier is made of pharmaceutical or cosmetic ingredients, known to persons skilled in the art, which are selected from stabilizing, suspending or gelling agents, that are devoid of the unwanted effects of fibroblasts and keratocytes toxicity and wound healing inhibition and irritation at the concentrations used at the application.

Most preferred stabilizing agents are hydrocolloids and mild non-ionic surfactants, which cause at most only minimal hemolysis.

Hydrocolloids are hydrophilic polymers, of vegetable, animal, microbial or synthetic origin, that generally contain many hydroxyl groups and may be polyelectrolytes. They are naturally present or added to control the functional properties of aqueous pharmaceutical and cosmetics. Most important amongst these properties are viscosity (including thickening and gelling) and water binding but also significant are many other properties, including emulsion stabilization, prevention of ice re-crystallization and organoleptic properties.

Preferred hydrocolloids are selected from the group consisting of Alginate, Cellulose and cellulose derivatives such as hydroxy methyl ethyl and propyl derivatives, Xanthan gum, Gum arabica, Carrageenan, Guar gum, Gelatin, Pectin, Starch, Carboxy-methylcellulose, Hyaluronic acid and Chitosan, Alginate, pullulan, polyvinyl pyrrolidone, polyvinyl alcohol, dextrin, pectin, chitin, collagen, gelatin, zein, gluten, starch and starch derivatives.

Preferred mild non-ionic surfactants are Sucrose esters and Sorbitan esters such as spans.

Example of unwanted ingredients that are irritating and wound healing inhibitors are pharmaceutical solvents such as ethyl-alcohol and stabilizers such as sodium lauryl sulphate or polyoxyethylene polymers derivatives, used extensively in medicine and cosmetics, but are avoided in the formulations and products of the present invention.

Ethanol, propylene glycol, dimethylsulfoxide, dimethylformamide, and Brij 96 have been shown to be cytotoxic to human keratinocyte and fibroblast cultures (Ponec et al. J Pharm Sci 1990 April; 79(4): 312-6) and are all inappropriate for use in the invented composition.

The anti-microbial drugs contemplated for use in the present invention are selected from the group consisting of antibiotics, anti-fungals, anti-protozoals and anti-virals. Antibiotics include but are not limited to: beta-lactams penicillins and cephalosporines, Macrolides, Licosamides, Aminoglicosides such as Gentamycin, Tetracyclines, Polypepetides such as Vancomycin, Sulfonamides, Flioroquinolones, chloramphenicol, nitrofurantoin and chlorhexidine. Anti-fulgals including but not limited to: Nystatine, Amphotericine B, Griseofulvine, Miconazole, Itraconazole, Fluconazole, Ketoconazole, Terbinafine, Silver Sulfadiazine, Flucytosine and Clotrimazole. Anti-protozoals include but arenot limited to: metronidazole, eflornithine, furazolidone, hydroxychloroquine, iodoquinol and pentamidine. Anti-virals include but are not limited to acyclovir, amantadine, famciclovir, ganciclovir, rimantadine and valacyclovir.

Antimicrobial agents are defined as organic chemicals that derive their antimicrobial activity through a chemical or physiochemical interaction with the microbial organisms. For example, Cetyl pyridinium chloride, triclosan, biguanides include the free bases or salts of alexidine, chlorhexidine, hexamethylene biguanides and their polymers, and combinations of the foregoing. The salts of alexidine and chlorbexidine can be either organic or inorganic and are typically gluconates, nitrates, acetates, phosphates, sulfates, halides and the like. The preferred biguanide is the hexamethylene biguanide commercially available from Zeneca, Wilmington, Del. under the trademark Cosmocil.™. CQ. Generally, the hexamethylene biguanide polymers, also referred to as polyaminopropyl biguanide (PAPB), have molecular weights of up to about 100,000.

An essential oil or volatile oil is a volatile mixture of esters, aldehydes, alcohols, ketones and terpenes, which is prepared from botanical materials or plant cell bio-mass from cell culture. Examples of essential oils include, but are not limited to, oil of cinnamon, prepared from the dried bark of the roots of Cinnamomum zeyloriaceae; cajeput oil, eucalyptus oil, prepared from the fresh leaves and branches of various species of Eucalyptus, such as E. globulus; fennel oil, prepared from dried ripe fruit of Foeniculum vulgare; geranium oil, prepared from the aerial parts of Pelargonium species; girofle oil, lavander oil, prepared from fresh flowering tops of Lavandula species such as Lavandula officinalis; lemon oil, obtained from the fresh peel of Citrus lemon; spearmint oil, prepared from the aboveground parts of fresh flowering Mentha species, such as M. spicata; myrte oil, origano oil, pine oil, rosemary oil, prepared from tops or leafy twigs of Rosmarinus officinalis; sarriette oil, thyme oil, prepared from the leaves and flowering tops of Thymus vulgaris; and tea-tree oil, obtained from the leaves of Melaleuca oltemifolia. Hypericum oil, Pinus, Star anise seeds oil, Lemon oil and Garlic oil (Allium sativum oil).

Also included in this class of essential oils are the key chemical components of the plant oils, which have been found to be the major constituents of the natural oil and which have in many cases identical activity and typical physical and chemical properties. These chemicals include, but are not limited to anethol, catechole, camphene, thymol, eugenol, eucalyptol, ferulic acid, farnesol, hinokitiol, tropolone, limonene, menthol, methyl salicylate, carvacol, terpineol, verbenone, berberine, ratanhiae extract, caryophellene oxide, citronella acid, curcumin, nerolidol and geraniol.

The composition of the present invention may further include a wound healing agent such as but not limited to Aloe vera dry extract, Herbals tannins, Echinacea extract, Comfrey extract, Allantoin, Turmeric dry extract, and recombinant growth factors. Hyaluronic acid, alginates and chitosans, which are also known to be a wound-healing agent may serve double functions, wound healing and major vehicle stabilizing excipient.

The liquid and/or semi-solid delivery system of the present invention may be used as is for application on the affected disease location, or can be provided as a concentrated formula to be diluted before use to obtain proper concentration of the bio-actives: the anti-microbial drug and the essential oil. Concentrated formulas are simpler to stabilize and achieve long shelf life and microbiological preservation, while producing a non-cytotoxic and wound healing formulation upon dilution before use.

Bactericidal ointments for the treatment of wounds are well known. Such ointments typically contain an antibiotic or an anti-bacterial agent in an inert vehicle or carrier, such as a paraffin base ointment or an oil-in-water emulsion. Antibiotics, which are Used, include gentamycin sulphate and neomycin sulphate, while anti-bacterial agents include cetrimide, chlorhexidine gluconate and silver sulphadiazine.

An inactive ingredient which is non-cytotoxic and which does not inhibit wound-healing is known per se and is selected from those chemical or pharmaceutical non-active ingredients used for stabilizing the formulation which do not show toxicity or killing effect to fibroblats and keratinocytes in in-vitro culture.

Non-irritating chemicals are those that do not cause local inflammatory reaction and which do not produce tissue destruction or irreversible change at the site of contact; the macroscopic manifestations of irritation are edema and erythema. Alcohol causes moderate skin irritation. Other common irritating ingredients are ionic surfactants, microbial preservatives and non-ionic PEO surfactants.

Bacterial multiple resistance to antibiotics is a major problem in modern medicine. Essential oils are potent anti-microbials with no reported major bacterial resistance. Combining anti-microbial drug and essential oil enables application of reduced drug concentration while keeping anti-microbial activity, hence improving therapeutic index and overcoming the multiple resistance problems.

Anhydrous bases are made of olefins, silicon or polyols and may be liquid or semi-solid. Examples of such polyols include, but are not restricted to polyethylene glycol, propylene glycol, polypropylene glycol, diethylene glycol, glycerine and ethylene glycol.

Liquid or semi-solid aqueous formulas at the final application concentration may include polyols only in limited quantities that should not produce cytotoxic product. High polyols concentration is possible according to the current invention, in cases of products that are diluted before use, in a way that the final application product is so much diluted as to not have cytotoxic or wound-healing inhibition effects.

Liquid or semi-solid compositions of the present invention may be further packaged in plastic bottles, tubes, aluminum tubes pressurized aerosol or foam or non pressurized aerosol or drops or glass bottles, as well as in any other conventional packaging and closure materials. Solid dosage forms may be shaped into small unit chips for periodontal pocket insertion or into confectionary or strips for oral mastication.

A preferred composition of the present invention is formulated in concentrate form for subsequent dilution before use. Precise dilution is obtained by using a dosing pump or any other dosing device such as droppers or measuring cups. A preferred method is a dual chamber packaging wherein the concentrate is placed in one compartment and the dilution aqueous medium is placed in the second compartment and two compartments or chambers are mixed together before use to obtain the desired composition and concentrations of the anti-microbial drug and the essential oil or oils composition, in an isotonic or moderately hypertonic product that do not comprise ingredients in concentrations that inhibit tissue healing.

The anti-microbial and essential oil mixture for treating mucous, wound infections and ulcers may also contain common pharmaceutical additives, such as but not limited to; flavors or sweeteners in oral use, anti-oxidants such as vitamin E or CoenzymeQ10 or colorant or emollients, as common in the pharmaceutical art, in such concentration that no cytotoxic effect is present in the method of application.

The preferred anti-microbial drug concentration is dictated from its USP-NF monograph, the PDR or instruction for use as approved by the regulatory agencies. Concentrated formulas may have for example 10 times the recommended use concentration, and are diluted accordingly, 10 times before use by medical team or patient, to obtain the desired drug concentration for application.

According to the present invention, finally diluted composition which is directly applied onto affected area should be isotonic or of low or moderate hyper-tonicity and not hypertonic. Examples of unwanted hypertonic compositions are: 70% Sorbitol or 10% Glycerin in final formulation.

An isotonic solution in medicine is one that can be mixed with body fluids without causing any disturbance that is about 280 to 320 milliosmolar. Moderate hypertonic is a solution with osmotic pressure of less than twice the isotonic pressure and preferably not higher than 50% of isotonic solution.

Essential oils, such as Thyme, Eucalyptus and Cinnamon oils are most preferably used at concentrations of 0.05% to 0.5%, whereas 10 times concentrated formula may contain 0.5% to 5% to be diluted 10 times before use to obtain desired final concentration for application. Mucositis treatment requires low essential oils concentration while aphtouse or mouth ulcers are treated with much higher concentration.

Preferred stabilizing agents include alginate, hyaluronic acid, chitosan, acacia, xanthan gum, locust bean gum, guar gum, cellulose derivatives and gelatin and the like, in amounts ranging from about 0.01 to about 10.0 wt. %, preferably about 0.2 to about 4 wt. %.

Preferred emulsifying agents include sucrose esters, sorbitan esters, polyglyceryl esters, lecithin, bentonite, veegum, and the like, in amounts ranging from about 0.01 to about 4 wt. %, preferably about 0.1 to about 1.0 wt. %.

Preferred thickening agents include methylcellulose, hydroxypropylmethyl cellulose, carboxy-methylcellulose, and the like, in amounts ranging from about 0.01 to about 10 wt. %, preferably about 0.1 to about 4 wt. %.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLES

### Example 1

| INGREDIENT | % w/w |
|---|---|
| Chlorhexidine gluconate 20% solution | 20 |
| Thyme oil | 0.5 |
| Cinammon oil | 0.5 |
| Aloe vera dry extract | 0.2 |
| Methyl cellulose 4000 | 4.0 |
| Sucrose ester (HLB 15) | 0.6 |
| Sweetener Stevia extract | 0.1 |
| MCT oil | 10.0 |
| Glycerin | To 100 |

This Mucositis mouthwash is a concentrated formula to be diluted with water before use by the patient. Precise twenty times dilution with water is enabled by using dosing pump or dual chamber device to obtain final Chlorhexidine gluconate concentration of 0.2%.

This Mucositis mouthwash was well tolerated even by severe mucositis developed after chemotherapy and irradiation therapy. Patients use it at-libidum in contrast to the unaccepted alcoholic marketed Chlorhexidine mouthwash, and report on reduced pains, improved swallowing and faster Mucositis healing.

### Example 2

### Mouth Ulcers (Aphtouse) Mouthwash

| INGREDIENT | % w/w |
|---|---|
| Acyclovir | 2.0 |
| Thyme oil | 0.5 |
| Tea Tree oil | 0.5 |
| Cinammon oil | 0.5 |
| Aloe vera dry extract | 0.1 |
| Echinacea glycerin extract | 1.0 |
| Propolis | 0.2 |
| Sodium Carboxymethylcellulose | 1.5 |
| Xanthan gum | 0.2 |
| Sucrose ester (HLB 15) | 0.4 |
| MCT oil | 0.5 |
| Glycerin | 4.0 |
| Water | To 100 |

This aphtouse formula was effective in treating recurrent aphtouse (mouth ulcers) conditions, reducing pain, shortening healing period and enabling comfortable eating including acidic orange juice, within 24 hour of aphtouse eruption and treatment.

### Example 3

### Vaginal Wash

| INGREDIENT | % w/w |
|---|---|
| Miconazole | 10.0 |
| MCT oil | 8.0 |
| Thyme oil | 0.5 |
| Lavender oil | 0.5 |
| Aloe vera dry extract | 0.2 |
| Methyl cellulose 4000 | 4.0 |
| Span 80 | 0.8 |
| Glycerin | To 100 |

This Vaginal wash formula should be diluted 10 times with water before use. It has been found effective in treating Vaginitis of yeast infection and well accepted.

### Example 4

### Vaginal Gel

| INGREDIENT | % w/w |
|---|---|
| Metronidazole | 1.0 |
| Thyme oil | 0.1 |
| Geranium oil | 0.1 |
| Aloe vera dry extract | 0.1 |
| Methyl cellulose 4000 | 4.0 |
| Xantan gum | 0.2 |
| Sucrose ester (HLB 15) | 0.6 |
| Water | To 100 |

### Example 5

### Anal Fissure Gel

| INGREDIENT | % w/w |
|---|---|
| Chlorhexidine gluconate 20% | 0.2 |
| Thyme oil | 0.2 |
| Eucalyptus oil | 0.2 |
| Hypericum oil | 0.2 |
| Echinacea dry extract | 0.2 |
| Chamomile dry extract | 0.2 |
| Sodium Carboxymethylcellulose | 1.2 |
| Span 80 | 0.5 |
| Water | To 100 |

This Anal fissure formula was effective in reducing pain, shortening healing period and treating anal fissures without use of steroids.

### Example 6

### Pressure Sore Wash

| INGREDIENT | % w/w |
|---|---|
| Chlorhexidine gluconate 20% | 10 |
| Thyme oil | 0.5 |
| Eucalyptus oil | 0.5 |
| Aloe vera dry extract | 0.2 |
| Alginate | 2.0 |
| Sucrose ester (HLB 15) | 0.6 |
| Glycerin | To 100 |

This pressure sore formula is concentrated and should be diluted with sterile water before use to obtain 0.2% Chlorhexidine gluconate wash solution.

### Example 7

### Pressure Sore Dressing

| INGREDIENT | % w/w |
|---|---|
| Chloramphenicol | 1.0 |
| Thyme oil | 0.1 |
| Eucaliptus oil | 0.1 |
| Alantoin | 1.0 |
| Aloe vera dry extract | 0.1 |
| Hyaluronic acid | 2.0 |
| Sucrose ester (HLB 15) | 0.2 |
| Water | To 100 |

### Example 8

### Acne Gel

| INGREDIENT | % w/w |
|---|---|
| Tetracycline | 2.0 |
| Geranium oil | 0.05 |
| Citronella oil | 0.05 |
| Aloe vera dry extract | 0.1 |
| Hyaluronic acid | 0.5 |
| Span 80 | 0.4 |
| Glycerin | 10.0 |
| Water | To 100 |

### Example 9

### Skin Ulcer Dressing

| INGREDIENT | % w/w |
|---|---|
| Silver sulfasalazine | 1.0 |
| Geranium oil | 0.05 |
| Thyme oil | 0.05 |
| Aloe vera dry extract | 0.1 |
| Chitosan | 4.0 |
| Span 80 | 0.2 |
| Water | To 100 |

This formula proved to be effective anti-microbial treatment for infected skin ulcers. Chitosan may require low pH for hydration.

### Example 10

### Ear Drops

| INGREDIENT | % w/w |
|---|---|
| Bacitracin | 1.0 |
| Eucalyptus oil | 0.25 |
| Thyme oil | 0.25 |
| Aloe vera dry extract | 0.1 |
| Hyaluronic acid | 0.2 |
| Span 80 | 0.2 |
| Carbopol 980 | 0.6 |
| Water | To 100 |

This ears drops formula proved to be effective anti-microbial treatment for infected Otitis externa and pain relief in difficult cases.

### Example 11

### Mouth Rinse to Treat Gingivitis

| INGREDIENT | % w/w |
|---|---|
| Cety pyridinium chloride | 2.0 |
| Eucalyptol | 3.68 |
| Thymol | 2.56 |
| Menthol | 1.68 |
| Methyl salycilate | 2.4 |
| Aloe vera dry extract | 0.1 |
| Hyaluronic acid | 0.2 |
| Span 80 | 0.2 |
| Pemulen TR1 | 0.2 |
| MCT oil | 2.0 |
| Glycerin | To 100 |

This anti gingivitis mouth rinse formula is diluted 20 times with water before use and is stable after reconstitution for couple of months and has been proved to be effective anti-gingivitis treatment in kids, diabetics and other conditions where use of alcohol prohibited or not recommended.

### Example 12

### Mouth Rinse to Treat Gingivitis

| INGREDIENT | % w/w |
|---|---|
| Cety pyridinium chloride | 2.0 |
| Eucalyptol | 0.4 |
| Thymol | 0.6 |
| Menthol | 1.2 |
| Methyl salycilate | 1.0 |
| Sucrose ester | 0.8 |
| Span 80 | 0.2 |
| Pemulen TR2 | 0.2 |
| MCT oil | 4.0 |
| Glycerin | To 100 |

This anti gingivitis mouth rinse formula is diluted 20 times with water before use and is stable after reconstitution for couple of months.

### Example 13

### Mouth Rinse for Dental Plaque Control

| INGREDIENT | % w/w |
|---|---|
| Sodium Fluoride | 1.0 |
| Eucalyptol | 0.4 |
| Thymol | 0.6 |
| Menthol | 1.2 |
| Methyl salycilate | 1.0 |
| Hammamelis dry extract | 0.1 |
| Sucrose ester | 0.8 |
| Pemulen TR2 | 0.2 |
| MCT oil | 2.0 |
| Propylene glycol | To 100 |

This anti gingivitis mouth rinse formula is diluted 20 times with water before use and is stable after reconstitution for couple of months.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A composition of matter for treating infected skin and mucousal membranes, said composition comprising:
a) at least one anti-microbial drug; and
b) at least one essential oil,
in combination with a substantially, alcohol-free carrier system, said carrier being selected from a liquid carrier or a semi-solid carrier, said carrier system being selected from isotonic system and a moderately hypertonic system.

2. A composition according to claim 1, wherein said carrier is substantially free of cytotoxic excipients and is free of excipients, which inhibit wound healing.

3. A composition according to claim 1, wherein the carrier is an aqueous formulation stabilized with a pharmaceutical or cosmetic excipent which does not inhibit wound healing.

4. A composition according to claim 1, wherein the carrier is an aqueous formulation stabilized with a hydrocolloid dispersing or gelling agent.

5. A composition according to claim 1, wherein the carrier is an aqueous formulation stabilized with a non-ionic surface-active stabilizing agent.

6. A composition according to claim 1, wherein the carrier is an aqueous formulation stabilized with a polysaccharide dispersing or gelling agent.

7. A composition according to claim 1, wherein the carrier is an aqueous formulation stabilized with a synthetic or semi-synthetic polymer as dispersing or gelling agent.

8. A composition according to claim 1, wherein the carrier is a liquid emulsion or suspension.

9. A composition according to claim 1, wherein the carrier is in the form of a semi-solid selected from the group consisting of a gel, an ointment and a cream.

10. A composition according to claim 1, wherein the carrier is an anhydrous base.

11. A composition according to claim 1, wherein the carrier is substantially free of alcohol and substantially free of organic solvents.

12. A composition according to claim 1, wherein the carrier is substantially free of irritating emulsifiers.

13. A composition according to claim 1, wherein the anti-microbial agent is selected from the group consisting of anti-bacterial drugs, anti-fungal drugs and anti-viral drugs.

14. A composition according to claim 1, wherein the essential oils are selected from pharmaceutical grade essential oils.

15. A composition according to claim 1 further comprising a wound-healing agent.

16. A composition according to claim 1, wherein said at least one anti-microbial drug and said at least one essential oil are concentrated and the composition is diluted with water before use to obtain the appropriate treatment concentration.

17. A method for preventing and or treatment of mucous and skin infection diseases selected form the group consisting of Mucositis, Gingivitis, Periodontitis, Vaginitis, Anal fissure, Skin ulcer, Otitis, Dermatoses comprising application of a formulation comprising at least one anti-microbial drug; and at least one essential oil, in combination with a substantially, alcohol-free carrier system, said carrier being selected from a liquid carrier or a semi-solid carrier, said carrier system being selected from isotonic system and a moderately hypertonic system.

18. A method according to claim 17, wherein the formulation is stabilized with a pharmaceutical or cosmetic hydrocolloid, which does not inhibit wound healing.

19. A method according to claim 17, wherein the formulation is stabilized with a pharmaceutical or cosmetic non-ionic surface-active agent, which does not inhibit wound healing.

20. A method according to claim 17, wherein the active ingredients in said formation are concentrated and the composition is diluted before use to obtain appropriate treatment concentration, which is not hypertonic.
